SEQUENCE LISTING (10 sequences)

SEQ ID NO: 1 - DNA, 22 bp, Artificial Sequence
  Synthetic primer
cacagaggtttcagtggtttgg

SEQ ID NO: 2 - DNA, 22 bp, Artificial Sequence
  Synthetic primer
gcaccagtagcaccatcatttc

SEQ ID NO: 3 - DNA, 22 bp, Artificial Sequence
  Synthetic primer
ctgaaattctgccatcctgaac

SEQ ID NO: 4 - DNA, 22 bp, Artificial Sequence
  Synthetic primer
ggattgccgtagctaaactgaa

SEQ ID NO: 5 - DNA, 22 bp, Artificial Sequence
  Synthetic primer
atcaacgttggtgctactgctt

SEQ ID NO: 6 - DNA, 22 bp, Artificial Sequence
  Synthetic primer
atctttagaggagccccaggta

SEQ ID NO: 7 - DNA, 22 bp, Artificial Sequence
  Synthetic primer
aagattggagagaagtgggacc

SEQ ID NO: 8 - DNA, 20 bp, Artificial Sequence
  Synthetic primer
gagcaaatggcaccgagata

SEQ ID NO: 9 - DNA, 20 bp, Artificial Sequence
  Synthetic primer
gagtcaacggatttggtcgt

SEQ ID NO: 10 - DNA, 20 bp, Artificial Sequence
  Synthetic primer
gacaagcttcccgttctcag

(19) United States
(12) Patent Application Publication
(10) Pub. No.: US 2019/0133922 A1
(43) Pub. Date: May 9, 2019

(54) Stem Cell-Derived Exosomes Containing a High Amount of Growth Factors

(71) Applicant: Kangstem Biotech Co., Ltd., Seoul (KR)

(72) Inventors: Kyung Sun KANG, Seoul (KR); Kwang Won SEO, Gyeonggi-do (KR); Yu Lee KIM, Seoul (KR); Yoon Jin KIM, Seoul (KR)

(21) Appl. No.: 16/069,540

(22) PCT Filed: Jan. 12, 2017
(86) PCT No.: PCT/KR2017/000420

§ 371 Date: Aug. 21, 2018

(30) Foreign Application Priority Data
Jan. 12, 2016 (KR) 10-2016-0003819

(51) Int. Cl. A61K 8/98; A61K 35/51; C12N 5/0775; A61K 9/00

(52) U.S. Cl. CPC A61K 8/98; A61K 9/0014; C12N 5/0665; A61K 35/51

(57) ABSTRACT

The present invention relates to exosomes with an increased amount of growth factors, which are obtained from stem cells cultured in a medium containing an epithelial growth factor (EGF) and/or a fibroblast growth factor (FGF). It is expected that the nano-sized exosomes isolated from a stem cell culture liquid can penetrate into the dermal layer of the skin and thereby increase their regenerative effect. In addition, since exosomes contain a large amount of various growth factors, they provide effects such as skin regeneration and anti-aging, promotion of collagen synthesis, hair growth, restoration of shrunken hair follicles, and wound healing through proliferation and activation of fibroblasts, which are skin component cells.

### TECHNICAL FIELD OF THE INVENTION

The present invention relates to exosomes with an increased amount of growth factors, which are obtained from stem cells cultured in a medium containing an epithelial growth factor (EGF) and/or a fibroblast growth factor (FGF), a method thereof, and use thereof.

### BACKGROUND OF THE INVENTION

With the increase of the aging population along with the improvement of living standards of modern people, interest and demand for functional cosmetics related to the prevention of aging, improvement of wrinkles, whitening, and ultraviolet rays are increasing. However, since basic or functional cosmetic products are mostly made of chemical materials, safety issues related to the human body have been constantly raised. Accordingly, there is a growing interest in cosmetic products containing natural and organic ingredients, and the market size of related products is also increasing.

For example, a cosmetic composition for improving skin conditions containing a human embryonic stem cell culture (KR Patent Application Publication No. 10-2015-0039343), a cosmetic composition for improving skin wrinkles or inhibiting skin aging containing a stem cell culture fluid derived from a mammal as an active ingredient (KR Patent Application Publication No. 10-1063299), etc. have been developed. However, there were ethical concerns over using human embryonic stem cells and problems in that the effect of human embryonic stem cells was insignificant, etc.

Mesenchymal stem cells are known to secrete cytokines and various growth factors such as epithelial growth factor and fibroblast growth factor, and have an important role of skin regeneration by promoting collagen production from fibroblasts. There is a growing interest in the development of cosmetics using stem cells with these properties, and in particular, research has been focused on the development of techniques for enhancing skin penetration of effective factors of stem cells.

### SUMMARY OF THE INVENTION

### Technical Problem

An object of the present invention is to provide a method for preparing exosomes with an increased amount of growth factors, which includes culturing stem cells in a medium that contains an epithelial growth factor, a fibroblast growth factor, and a combination thereof.

Another object of the present invention is to provide exosomes with an increased amount of growth factors, prepared by the method described above.

Still another object of the present invention is to provide a composition for increasing the amount of growth factors comprised in exosomes of stem cells, which contains an epithelial growth factor, a fibroblast growth factor, or a combination thereof.

Still another object of the present invention is to provide a cosmetic composition for improving skin conditions, which contains the exosomes as an active ingredient.

Still another object of the present invention is to provide a quasi-drug composition for improving skin conditions, which contains the exosomes as an active ingredient.

Still another object of the present invention is to provide a pharmaceutical composition for healing wounds, which contains the exosomes as an active ingredient.

### Advantageous Effects of the Invention

The exosomes obtained from umbilical cord blood-derived mesenchymal stem cells (hereinafter, UCB-MSCs) according to the present invention contain a higher level of EGF that is effective in skin regeneration, formation of hair follicles, wound healing, etc. compared to exosomes of different origins. Due to the particular lipid bilayer structure, exosomes can deliver EGF to the dermal layer of the skin, and thus, exosomes can be effectively used for the improvement of skin conditions (e.g., skin regeneration, anti-aging effect, increase of collagen synthesis, hair growth, restoration of shrunken hair follicles, etc.), and wound healing.

### BRIEF DESCRIPTION OF THE DRAWINGS

FIG. 1 shows images showing the results of a human growth factor antibody array of exosomes isolated from various cell culture media, in which CTL (control group) represents a culture of UCB-MSCs before the isolation of exosomes; “UCB-MSC exosome” represents exosomes isolated from the UCB-MSC culture; “AD-MSC exosome” represents exosomes isolated from a culture of human adipose-derived mesenchymal stem cells (AD-MSCs); and “BM-MSC exosome” represents exosomes isolated from a culture of human bone marrow derived mesenchymal stem cells (BM-MSCs).

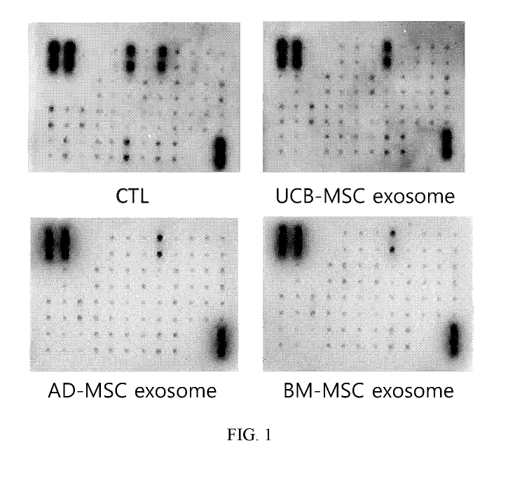

FIG. 2A shows graphs confirming the major growth factors of exosome isolated from various cell cultures, in which the amounts of various growth factors contained in exosomes isolated from the CTL (control group), UCB-MSC, AD-MSC, and BM-MSC cultures were compared.

FIG. 2B shows a graph confirming the major growth factors isolated from various cell cultures, in which the amounts of major growth factors contained in exosomes isolated from UCB-MSC, AD-MSC, and BM-MSC cultures excluding the CTL culture were compared.

FIG. 3 shows graphs comparing the amounts of major growth factors in exosomes according to culture conditions of UCB-MSCs. UCB-MSCs were cultured under the conditions where both EGF and FGF were present or absent. Specifically, FIG. 3A shows images illustrating the results of a human growth factor antibody array of exosomes, and FIG. 3B shows graphs illustrating the amounts of growth factors contained in the above exosomes.

FIG. 4 shows graphs illustrating the effect of increasing the expression level of the extracellular matrix (ECM) with regard to human dermal fibroblasts (HDFs) of exosomes isolated from the UCB-MSC culture. For the ECM, collagen type I, collagen type III, elastin, and fibronectin were used.

### DETAILED DESCRIPTION OF THE INVENTION

### Best Mode

To achieve the above objects, a first aspect of the present invention provides a method for preparing exosomes with an increased amount of growth factors, which includes culturing stem cells in a medium containing an epithelial growth factor, a fibroblast growth factor, and a combination thereof.

A second aspect of the present invention provides exosomes with an increased amount of growth factors, which were prepared by the preparation method of the first aspect.

A third aspect of the present invention provides a composition for increasing the amount of growth factors in exosomes of stem cells containing an epithelial growth factor, a fibroblast growth factor, and a combination thereof.

A fourth aspect of the present invention provides a cosmetic composition for improving skin conditions containing the exosomes as an active ingredient.

A fifth aspect of the present invention provides a quasi-drug composition for improving skin conditions containing the exosomes as an active ingredient.

A sixth aspect of the present invention provides a pharmaceutical composition for healing wounds containing the exosomes as an active ingredient.

Hereinafter, the present invention will be described in detail.

Exosomes are cell-derived vesicles consisting of a lipid bilayer, and they are secreted extracellularly in a state containing cell-specific proteins, RNAs, etc., and thereby transfer these proteins, RNAs, etc. to other cells.

The proteins contained in exosomes are protected by phospholipids in the form of a cell membrane, and thus their activities are not readily lost by proteases, etc., and these proteins can therefore stably perform their functions compared to soluble proteins. Additionally, the structure of a lipid bilayer is easily fused with a cell membrane, and thus the materials contained in exosomes can be efficiently delivered to cells (Lai, R. C. et al., Biotechnol. Adv., 5, 543 to 551, 2013).

Exosomes may be obtained from a cell culture after culturing cells. The processes of isolating and detecting exosomes are sophisticated.

Additionally, exosomes contain RNAs, proteins, lipids, and metabolites which reflect the cell types from which the corresponding exosomes are derived. Exosomes may contain various molecule-constituting elements (e.g., proteins and RNAs) from which the corresponding exosomes are derived. There are various exosome protein compositions depending on the cells and tissues from which the corresponding exosomes are derived, but most exosomes contain a set of evolutionarily preserved common protein molecules.

Meanwhile, the production and amounts of exosomes may be affected by the molecular signals received by exosome-producing cells.

Accordingly, the present inventors cultured mesenchymal stem cells derived from bone marrow, fats, and umbilical cord blood using a medium containing epithelial growth factor and/or fibroblast growth factor (FGF) and analyzed the exosomes isolated from their cultures, and as a result, they have discovered that exosomes containing various growth factors involved in cell growth (e.g., epithelial growth factor (EGF), vascular endothelial growth factor (VEGF), transforming growth factor (TGF), hepatocyte growth factor (HGF), fibroblast growth factor (FGF), insulin-like growth factor (IGF), and platelet-derived growth factor (PDGF)) in large amounts were produced, and have also discovered that EGF, which mainly regulates cell growth, was contained in the largest amount as the representative growth factor among the various growth factors. Additionally, they have discovered that mesenchymal stem cells derived from UCB-MSCs contained more diverse growth factors compared to those derived from bone marrow and fats. The present invention is based on such discoveries.

Since the exosomes isolated from stem cell cultures are nano-sized, they are expected to penetrate into the dermal layer of the skin and thereby increase the skin regeneration effect. Additionally, since these exosomes contain various growth factors in large amounts, they can provide effects such as skin regeneration, anti-aging, increase of collagen synthesis, hair growth, restoration of shrunken hair follicles, and wound healing through proliferation and activation of fibroblasts, which are skin-constituting cells.

Accordingly, the present invention provides a method for preparing exosomes with an increased amount of growth factors, which includes a two-step process of culturing stem cells in a medium containing EGF, FGF, or a combination thereof; and isolating exosomes from the stem cell culture. The present invention also provides exosomes with an increased amount of growth factors.

In particular, the concentrations of EGF and FGF contained in the medium may be in a range of 10 pg/mL to 10 μg/mL, but the concentrations are not limited thereto.

Additionally, the present invention provides a composition for increasing the amount of growth factors containing EGF, FGF, or a combination thereof.

The exosomes according to the present invention have a higher amount of growth factors compared to those obtained from the stem cells cultured in a medium not containing EGF and/or FGF.

Additionally, the exosomes may be produced or used in the form of a culture which contains exosomes, or may be produced or used in the form of the culture where cells are removed.

In a specific embodiment of the method for preparing exosomes according to the present invention, the method includes culturing stem cells in a medium containing EGF and/or FGF and secreting EGF-containing exosomes extracellularly; and isolating exosomes from the stem cell medium (culture).

The present invention is characterized in that a medium containing EGF and/or FGF is used for the production of exosomes where the stem cells contain EGF or various growth factors in large amounts. The medium may be a serum-free medium. For example, the medium may be be Dulbecco's Modified Eagle's Medium (DMEM) containing EGF and/or FGF.

The exosomes obtained using the medium according to the present invention may contain two or more kinds of growth factors, and the growth factor with the highest amount among them may be EGF.

Alternatively, the exosomes obtained using the medium according to the present invention may contain an epithelial growth factor (EGF); and at least one selected from the group consisting of vascular endothelial growth factor (VEGF), transforming growth factor (TGF), hepatocyte growth factor (HGF), fibroblast growth factor (FGF), insulin-like growth factor (IGF), and platelet-derived growth factor (PDGF).

Further, the exosomes of the present invention may further contain molecules that reflect the origin of stem cells where the exosomes were produced and secreted.

In the present invention, the stem cells that produce exosomes may be adult stem cells.

Adult stem cells are undifferentiated cells that are destined to be differentiated into cells of a particular tissue when necessary. Unlike the embryonic stem cells extracted from a human embryo, adult stem cells are extracted from grown body tissues, and thus, adult stem cells have an advantage in that ethical issues can be avoided. In the present invention, adult stem cells may be derived from umbilical cord, umbilical cord blood, bone marrow, fat, muscle, nerve, skin, amnion, or placenta, and more specifically, from umbilical cord blood, but the origins of adult stem cells are not limited thereto.

Additionally, the adult stem cells may be mesenchymal stem cells, mesenchymal stromal cells, or multipotent stem cells, but the adult stem cells are not limited thereto.

EGF is known to play an important role in skin regeneration by promoting proliferation of fibroblasts and collagen synthesis (Stanley Cohen, Developmental Biology 12, 394 to 407, 1965; A. Colige et al., Journal of Cellular Physiology 145:450 to 457, 1990). It has been suggested that EGF may be able to act as a factor to induce the formation of new hair follicles (Moo Yeol Hyun et al., International Wound Journal, 2014, doi: 10.1111/iwj.12354.) and has an excellent effect for wound healing (Hardwicke J et al., Surgeon, 2008 June; 6(3):172 to 177.). Additionally, exosomes are considered as ideal vesicles for drug delivery because they can transfer materials across a cell membrane. Such a lipid bilayer vesicle system is thought to be one of the most effective strategies for delivering drugs to the dermal layer of the skin because the system can overcome the problem of skin penetration (Saahil Arora et al., Asian Journal of Pharmaceutics, 6, 4, 237 to 244, 2012).

Therefore, the exosomes according to the present invention may be used as an active ingredient in a cosmetic composition for improving skin conditions, a quasi-drug composition for improving skin conditions, a composition for skin application, or a pharmaceutical composition for wound healing.

As used herein, the term “improvement of skin conditions” may refer to skin regeneration, improvement of skin elasticity, prevention or improvement of skin wrinkles, prevention or improvement of skin aging, growth of hair follicles, and/or restoration of shrunken

In particular, the term “improvement” refers to all activities that at least reduce the parameters associated with alleviation or treatment of conditions, e.g., the degree of symptoms.

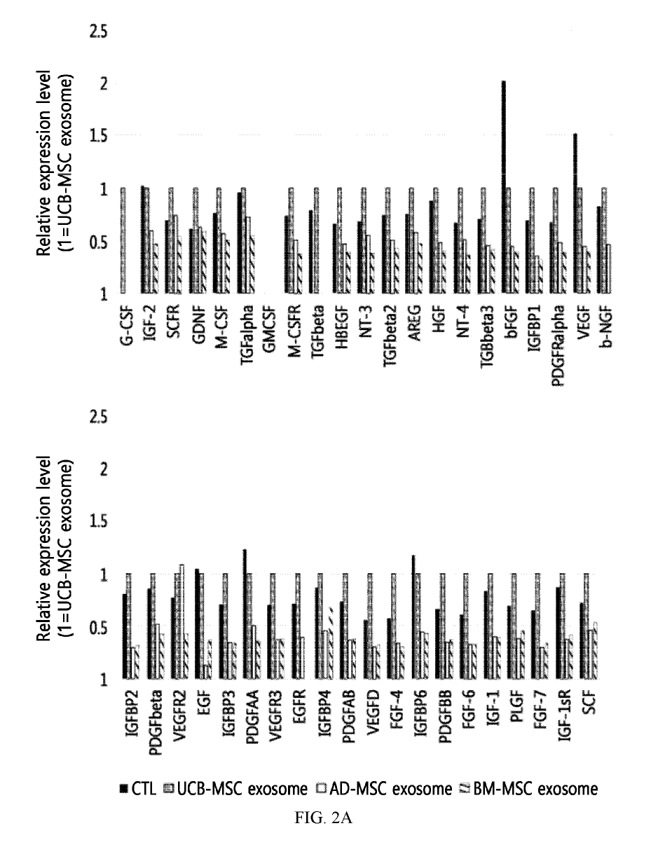

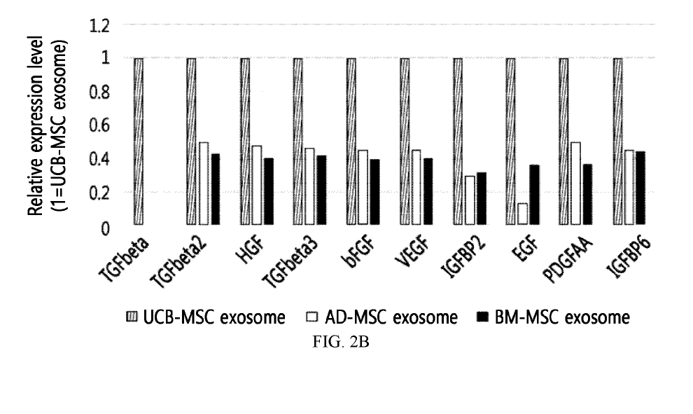

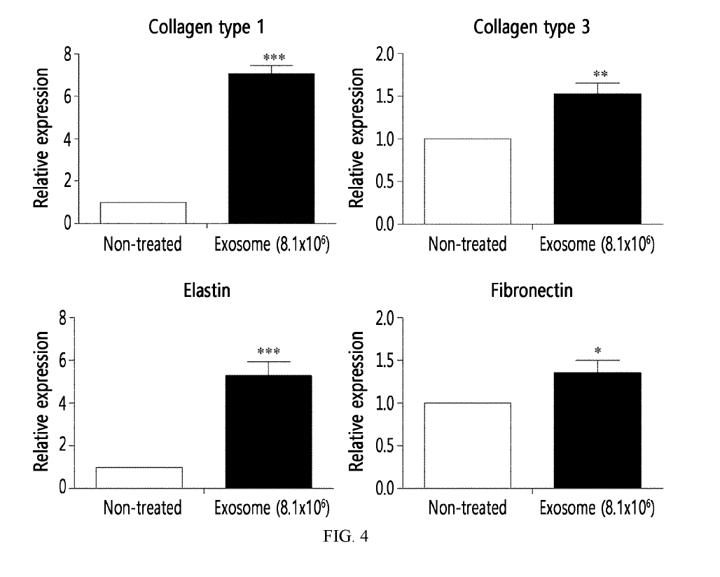

In a specific embodiment of the present invention, it was confirmed that the exosomes isolated from a UCB-MSC culture contained various growth factors (e.g., EGF, VEGF, TGF, HGF, FGF, IGF, PDGF, etc.) at a level higher than those isolated from the cultures of mesenchymal stem cells derived from fat tissue or mesenchymal stem cells derived from bone marrow, and in particular, that EGF was contained in a large amount (FIG. 2). Additionally, it was confirmed that the exosome-treated human skin fibroblasts showed a high level of expression of ECM proteins involved in skin elasticity (FIG. 4). Since the growth factors (e.g., EGF, etc.) promote the proliferation of fibroblasts (i.e., skin-constituting cells), cell migration and collagen synthesis, etc., it is suggested that the exosomes isolated from UCB-MSCs will be able to exhibit effects such as skin regeneration, improvement of skin elasticity, prevention or improvement of skin wrinkles, prevention or improvement of skin aging, growth of hair follicles, or restoration of shrunken hair follicles.

Meanwhile, the cosmetic composition according to the present invention may be prepared into a formulation selected from the group consisting of a solution, an ointment for external use, a cream, a foam, a nutrition emollient, a soft emollient, a fragrance, a pack, a soft water, an emulsion, a makeup base, an essence, a soap, a liquid cleansing agent, a bath preparation, a sunscreen cream, a sun oil, a suspension, a paste, a gel, a lotion, powders, a surfactant-containing cleansing agent, an oil, a powder foundation, an emulsion foundation, a wax foundation, a patch, and a spray, but the formulation is not limited thereto.

The cosmetic composition according to the present invention may further contain at least one kind of a cosmetically acceptable carrier which is mixed in a general skin cosmetic, and as a conventional component, for example, a fat component, water, a surfactant, a humectants, a low grade alcohol, a thickening agent, a chelating agent, a pigment, a preservative, a fragrance, etc. may be appropriately mixed, but the cosmetically acceptable carrier is not limited thereto.

The cosmetically acceptable carrier to be included in the cosmetic composition of the present invention may vary according to the formulation of the cosmetic composition.

When the formulation of the present invention is an ointment, a paste, a cream, or a gel, as the carrier component, an animal oil, a vegetable oil, wax, paraffin, starch, tragacanth, a cellulose derivative, polyethylene glycol, silicone, bentonite, silica, talc, zinc oxide, etc. may be used, but the carrier component is not limited thereto. The carrier component may be used alone or in a combination of two or more.

When the formulation of the present invention is a powder or spray, as the carrier component, lactose, talc, silica, aluminum hydroxide, calcium silicate, polyamide powder, etc. may be used, and in particular, when the formulation is a spray, a propellant such as chlorofluorohydrocarbons, propane/butane, or dimethyl ether may be used, but the carrier component is not limited thereto. These carrier components may be used alone or in a combination of two or more.

When the formulation of the present invention is a solution or emulsion, as the carrier component, a solvent, a solubilizing agent, an emulsifying agent, etc. may be used, for example, water, glycerin, ethanol, isopropanol, ethyl carbonate, ethyl acetate, benzyl alcohol, benzyl benzoate, propylene glycol, 1,3-butylglycol oil, etc. may be used, and particularly, cottonseed oil, peanut oil, corn seed oil, olive oil, castor oil and sesame oil, glycerol aliphatic ester, polyethylene glycol or fatty acid ester of sorbitan may be used, but the carrier component is not limited thereto. These carrier components may be used alone or in a combination of two or more.

When the formulation of the present invention is a suspension, as the carrier component, a liquid diluent (e.g., water, glycerin, ethanol, or propylene glycol), a suspending agent (e.g., ethoxylated isostearyl alcohol, polyoxyethylene sorbitol ester, and polyoxyethylene sorbitan ester), microcrystalline cellulose, aluminum metahydroxide, bentonite, agar, tragacanth, etc. may be used, but the carrier component is not limited thereto. These carrier components may be used alone or in a combination of two or more.

When the formulation of the present invention is a soap, as the carrier component, an alkali metal salt of a fatty acid, a hemiester salt of a fatty acid, a fatty acid protein hydrolysate, an isethionate, a lanolin derivative, an aliphatic alcohol, a vegetable oil, a glycerol, a saccharide, may be used, but the carrier component is not limited thereto. These carrier components may be be used alone or in a combination of two or more.

In the cosmetic composition of the present invention, the exosomes may be contained in an amount of 0.0001 wt % to 50 wt %, and more specifically 0.0005 wt % to 10 wt % relative to the total weight of the cosmetic composition. When the exosomes are contained within the above range, the cosmetic composition has advantages in that it has excellent effects of improving skin conditions and stabilizing the formulation of the composition.

As used herein, the term “quasi-drug” refers to products used for the purpose of diagnosis, cure, improvement, alleviation, treatment, or prevention of diseases of humans or animals, excluding those which are less active than pharmaceutical products or those used for pharmaceutical purposes. Those products which are used for the treatment or prevention of diseases of humans or animals, products that exhibit mild actions on the human body, or those which do not directly act on the human body are included.

The quasi-drug composition of the present invention may be prepared in the form of formulations selected from the group consisting of a body cleanser, foam, a soap, a mask, an ointment, a cream, a lotion, an essence, and a spray, but the formulations are not limited thereto.

The “composition for skin application” and “composition for wound healing” may be pharmaceutical compositions.

Accordingly, when these compositions are pharmaceutical compositions, they may further contain a pharmaceutically acceptable carrier, in addition to containing exosomes as an active ingredient.

As used herein, the term “pharmaceutically acceptable” means that when a compound is administered, it can be conventionally used in the pharmaceutical field without stimulating and inhibiting biological activities and characteristics of the compound.

The dose amount may be a pharmaceutically effective amount for the improvement of skin conditions. As used herein, the term “pharmaceutically effective amount” refers to an amount sufficient for the treatment of diseases at a reasonable benefit/risk ratio applicable to a medical treatment, and the level of the effective dose may be determined based on the factors including type of subject and severity of illness, age, sex, type of disease, drug activity, drug sensitivity, administration time, administration route, excretion rate, duration of treatment, factors including drugs to be used simultaneously in combination, and other factors well known in the medical field. Additionally, the effective amount may vary depending on administration route, use of excipients, and possibility of use in combination with other agents, as acknowledged to those skilled in the art.

In the present invention, the kind of the carrier is not particularly limited and any carrier conventionally used in the art may be used. Specific examples of the carrier may include saline, sterile water, Ringer's solution, buffered saline, albumin injection solution, lactose, dextrose, sucrose, sorbitol, mannitol, xylitol, erythritol, maltitol, maltodextrin, glycerol, ethanol, etc., but the carrier is not limited thereto. These carrier components may be used alone or in a combination of two or more.

Additionally, the composition may be used by adding other pharmaceutically acceptable additives (e.g., excipients, diluents, antioxidants, buffers, bacteriostats, etc.) thereto, if necessary, and a filler, an extender, a humectant, a disintegrant, a dispersant, a surfactant, a binder, a lubricant, etc. may additionally be used.

Additionally, still another aspect of the present invention provides a method for improving skin conditions, which includes administering to a subject exosomes with an increased amount of growth factors, which were obtained from stem cells cultured in a medium containing EGF and/or FGF.

As used herein, the term “subject” refers to all animals including mammals such as mice, cattle, humans, etc.

In the method for improving skin conditions of the present invention, the composition may be administered by intravenous administration, intraperitoneal administration, intramuscular administration, transdermal administration, subcutaneous administration, etc., and additionally, the composition may be administered by applying or spraying the composition to the skin, but the administration method is not limited thereto.

Hereinafter, the present invention will be described in more detail with reference to the following Examples. However, these Examples are for illustrative purposes only and the scope of the invention is not limited by these Examples only.

### Example 1. Cultivation of Mesenchymal Stem Cells and Collection of Culture

Human umbilical cord blood-derived mesenchymal stem cells (UCB-MSCs), human adipose-derived mesenchymal stem cells (AD-MSCs), and human bone marrow-derived mesenchymal stem cells (BM-MSCs) were cultured under the following conditions.

As a serum-free medium, UCB-MSCs, AD-MSCs, and BM-MSCs were dispensed at a concentration of 2×105 cells into each T25 flask, where Dulbecco's Modified Eagle's Medium (DMEM) was contained without a pH indicator (e.g., phenol red, etc.), and cultured in a 37° C. incubator with 5% CO2 for 2 days. Then, the existing culture was removed and the cells were washed with PBS, and the medium was replaced with DMEM containing EGF and FGF or DMEM not containing EGF and FGF, in a 2.5-fold volume of the existing culture. The cells were cultured for 4 days in the replaced medium, and thereby the UCB-MSC, AD-MSC, and BM-MSC cultures were obtained.

### Example 2. Isolation of Exosomes from Cultures

Each of the cultures obtained in Example 1 was transferred into a 50 mL tube and centrifuged at 2,000 rpm for 5 minutes. The resulting pellet of each cell culture was discarded and only the supernatant of each cell culture was collected and transferred into a 50 mL tube and centrifuged at 2,000×g for 30 minutes. Each supernatant separated by centrifugation was again transferred into a fresh 50 mL tube.

Then, the Exosome Isolation Reagent (Invitrogen, Cat. No. 4478359) was added to each of the separated supernatants in a ratio of 500 μL:1 mL and incubated at 4° C. After 24 hours, each mixture was centrifuged at 10,000×g for 60 minutes and each pellet formed was resuspended in 1×PBS to prepare isolated exosomes.

### Experimental Example 1. Analysis of Active Ingredients in Isolated Exosomes

### Experimental Example 1-1. Confirmation of Presence of Active Ingredient

To confirm the presence of ingredients of exosomes isolated from each culture of stem cells, the exosomes were subjected to the Human Growth Factor Antibody Array (RayBiotech, Cat. No. AAH-GF-1-8). The components present in the exosomes were confirmed by the ARRAY MAP of Table 1 below, and the relative amount of each growth factor was confirmed by comparative analysis of the ingredients with the positive control spot through Image J.

TABLE 1
ARRAY MAP

| | A | B | C | D | E | F | G | H | I | J | K | L |
|---|---|---|---|---|---|---|---|---|---|---|---|---|
| 1 | POS | POS | NEG | NEG | AREG | bFGF | b-NGF | EGF | EGFR | FGF-4 | FGF-6 | FGF-7 |
| 2 | POS | POS | NEG | NEG | AREG | bFGF | b-NGF | EGF | EGFR | FGF-4 | FGF-6 | FGF-7 |
| 3 | G-CSF | GDNF | GM | HB | HGF | IGFBP | IGFBP | IGFBP | IGFBP | IGFBP | IGF-1 | IGF-1 |
| | | | CSF | EGF | | 1 | 2 | 3 | 4 | 6 | | sR |
| 4 | G-CSF | GDNF | GM | HB | HGF | IGFBP | IGFBP | IGFBP | IGFBP | IGFBP | IGF-1 | IGF-1 |
| | | | CSF | EGF | | 1 | 2 | 3 | 4 | 6 | | sR |
| 5 | IGF-2 | M-CSF | M-CSF | NT-3 | NT-4 | PDGF R | PDGF R | PDGF | PDGF | PDGF | PLGF | SCF |
| | | | R | | | alpha | beta | AA | AB | BB | | |
| 6 | IGF-2 | M-CSF | M-CSF | NT-3 | NT-4 | PDGF R | PDGF R | PDGF | PDGF | PDGF | PLGF | SCF |
| | | | R | | | alpha | beta | AA | AB | BB | | |
| 7 | SCF | TGF | TGF | TGF | TGF | VEGF | VEGF | VEGF | VEGF | BLANK | BLANK | POS |
| | R | alpha | beta | beta 2 | beta 3 | | R2 | R3 | D | | | |
| 8 | SCF | TGF | TGF | TGF | TGF | VEGF | VEGF | VEGF | VEGF | BLANK | BLANK | POS |
| | R | alpha | beta | beta 2 | beta 3 | | R2 | R3 | D | | | |

POS = Positive Control Spot
NEG = Negative Control Spot
BLANK = Blank Spot

Specifically, the ingredients contained in UCB-MSC CM (CTL), which is a culture of UCB-MSCs themselves; exosomes isolated from the culture of UCB-MSCs (hereinafter, “UCB-MSC exosome”); exosomes isolated from the culture of AD-MSCs (hereinafter, “AD-MSC exosome”); and exosomes isolated from the culture of BM-MSCs (hereinafter, “BM-MSC exosome”) were compared.

As a result, it was confirmed that various growth factors are present in exosomes, as shown in FIG. 2A. In particular, it was confirmed that epithelial growth factor (EGF), which regulates cell growth, is most dominant in exosomes. Additionally, it was confirmed that the “UCB-MSC exosome” contained various growth factors at higher levels than the “AD-MSC exosome” or “BM-MSC exosome”.

Additionally, among the growth factors, epithelial growth factor (EGF), fibroblast growth factor (FGF), vascular endothelial growth factor (VEGF), transforming growth factor (TGF), platelet-derived growth factor (PDGF), hepatocyte growth factor (HGF), etc., which are associated with cell growth, were examined. As a result, it was confirmed that the “UCB-MSC exosome” contained a greater amount of growth factors compared to the exosomes obtained from cultures of stem cells derived from sources other than umbilical cord blood, and particularly, EGF was contained in a significantly high amount, as shown in FIG. 2B.

### Experimental Example 2. Comparison of Active Ingredients in Exosomes According to Culture Conditions

To develop a method for further increasing the amount of active ingredients in exosomes, the UCB-MSCs were cultured under the conditions where the growth factors EGF and FGF were added or not added, and the amount of active ingredients in exosomes isolated from the cultured UCB-MSCs was compared through the Human Growth Factor Antibody Array.

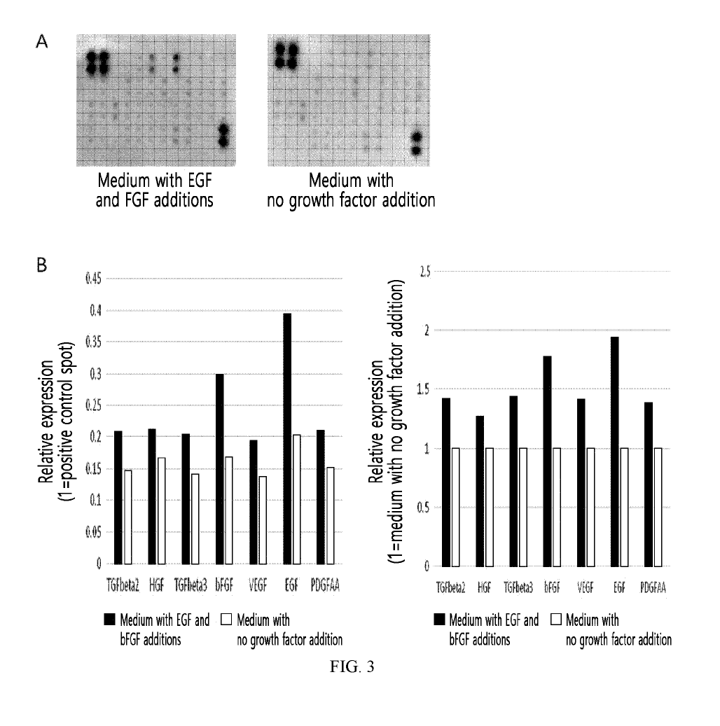

As a result, it was confirmed that the “UCB-MSC exosome”, under the conditions of a medium where EGF and FGF were added, showed an increase in the amounts of all of the growth factors (e.g., transforming growth factor-beta 2 (TGFβ2), HGF, transforming growth factor-beta 3 (TGFβ3), bFGF, VEGF, EGF, platelet-derived growth factor-AA (PDGFAA), etc.) by 1.5- to 2-fold, as shown in FIG. 3. From these results, it was confirmed that when UCB-MSCs were cultured using EGF and FGF, the amount of exosomes derived from the cells described above could be further increased.

### Experimental Example 3. Confirmation of Effect of Isolated Exosomes on Improvement of Skin Conditions

As it was confirmed in Experimental Example 1 that the “UCB-MSC exosome” of the present invention contained a significant amount of exosomes, the present inventors made an attempt to examine the effect of the “UCB-MSC exosome” on the improvement of skin conditions.

Specifically, human dermal fibroblasts (HDF) were treated with the “UCB-MSC exosome” isolated according to Example 2, and the expression level of the extracellular matrix (ECM) of these cells was confirmed by qPCR. HDF was inoculated into each well of a 6-well plate at a density of 2×105 cells and cultured for 24 hours. Then, the exosomes isolated according to Example 2 were added thereto at a density of 8×106 cells, cultured for 48 hours, and total RNA was obtained from each cultured HDF, and each cDNA was synthesized therefrom. The mRNA levels of collagen type I, collagen type III, elastin, and fibronectin were compared by performing real-time qPCR using each synthesized cDNA as a template along with the primers described in Table 2. In particular, GAPDH was used as the internal control group.

TABLE 2

| Name | Primer | Category |
|---|---|---|
| Collagen Type I | F: 5′-cacagaggtttcagtggtttgg-3′ | SEQ ID NO: 1 |
| | R: 5′-gcaccagtagcaccatcatttc-3′ | SEQ ID NO: 2 |
| Collagen Type III | F: 5′-ctgaaattctgccatcctgaac-3′ | SEQ ID NO: 3 |
| | R: 5′-ggattgccgtagctaaactgaa-3′ | SEQ ID NO: 4 |
| Elastin | F: 5′-atcaacgttggtgctactgctt-3′ | SEQ ID NO: 5 |
| | R: 5′-atctttagaggagccccaggta-3′ | SEQ ID NO: 6 |
| Fibronectin | F: 5′-aagattggagagaagtgggacc-3′ | SEQ ID NO: 7 |
| | R: 5′-gagcaaatggcaccgagata-3' | SEQ ID NO: 8 |
| GAPDH | F: 5′-gagtcaacggatttggtcgt-3′ | SEQ ID NO: 9 |
| | R: 5′-gacaagcttcccgttctcag-3′ | SEQ ID NO: 10 |

As a result, it was confirmed that when the “UCB-MSC exosome” was treated to ECM proteins related to skin elasticity (e.g., collagen, elastin, fibronectin, etc.), the expression levels of these ECM proteins were significantly increased compared to those of the “Non-treated” test group, as shown in FIG. 4. It was confirmed that collagen type I showed an increase of the expression level by about 7-fold, collagen type III by about 1.5-fold, elastin by about 5-fold, and fibronectin by about 1.3-fold. From these results, it was confirmed that the “UCB-MSC exosome” according to the present invention can exhibit the effect of improving skin elasticity.

Accordingly, the exosomes isolated from the culture of umbilical cord blood-derived stem cells contain a significantly higher amount of growth factors compared to those isolated from the cultures of stem cells derived from adipose tissue or bone marrow, and these exosomes, in a state of containing a large amount of various growth factors, can penetrate into the dermal layer of the skin, and thus they can exhibit effects such as skin regeneration, anti-aging, increase of collagen synthesis, hair growth, restoration of shrunken hair follicles, and wound healing through proliferation and activation of fibroblasts, which are skin-constituting cells.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method for preparing exosomes with an increased amount of growth factors, comprising culturing stem cells in a medium that comprises an epithelial growth factor, a fibroblast growth factor, and a combination thereof.

2. The method of claim 1, wherein the stem cells are adult stem cells derived from umbilical cord, umbilical cord blood, bone marrow, fat, muscle, nerve, skin, amnion, or placenta.

3. The method of claim 1, wherein the stem cells are stem cells derived from umbilical cord blood.

4. The method of claim 1, wherein the medium is a serum-free medium.

5. The method of claim 1, wherein the medium is Dulbecco's Modified Eagle's Medium (DMEM) comprising an epithelial growth factor, a fibroblast growth factor, or a combination thereof.

6. The method of claim 1, wherein the exosomes comprise at least two kinds of growth factors, and the epithelial growth factor has the highest expression level among the growth factors.

7. The method of claim 1, wherein the exosome comprises an epithelial growth factor; and at least one selected from the group consisting of a vascular endothelial growth factor, a transforming growth factor, a hepatocyte growth factor, a fibroblast growth factor, an insulin-like growth factor, and a platelet-derived growth factor.

8. The method of claim 1, wherein the method comprises isolating exosomes from the culture of adult stem cells.

9. Exosomes with an increased amount of growth factors prepared by the method of any one of claim 1.

10. A method of increasing the amount of growth factors comprised in exosomes of stem cells, comprising culturing stem cells in a medium that comprises an epithelial growth factor, a fibroblast growth factor, or a combination thereof.

11. A method of improving skin conditions comprising the step of administering to a subject a composition including the exosomes of claim 9 as an active ingredient.

12. The method of claim 11, wherein the improvement of skin conditions is at least one selected from the group consisting of skin regeneration, improvement of skin elasticity, prevention or improvement of skin wrinkles, prevention or improvement of skin aging, hair growth, and restoration of shrunken hair follicles.

13. The method of claim 11, wherein the composition is for application to the skin.

14. (canceled)

15. A method of healing wounds comprising the step of administering to a subject the exosomes of claim 9 as an active ingredient.
